(19) United States
(12) Patent Application Publication
(10) Pub. No.: US 2023/0405258 A1
(43) Pub. Date: Dec. 21, 2023

(54) Water Tank Installation Structure and Ventilation Therapy Device

(71) Applicant: BMC(Tianjin)Medical Co., Ltd., Tianjin (CN)

(72) Inventors: Lijun LIU, Beijing (CN); Zhi ZHUANG, Beijing (CN)

(21) Appl. No.: 18/038,095

(22) PCT Filed: Sep. 22, 2021
(86) PCT No.: PCT/CN2021/125729

§ 371 Date: May 22, 2023

(30) Foreign Application Priority Data
Nov. 25, 2020 (CN) 202011342914.8

(51) Int. Cl. A61M 16/16; A61M 16/10

(52) U.S. Cl. CPC A61M 16/162; A61M 16/109

(57) ABSTRACT

Provided are a water tank installation structure and a ventilation therapy device. The water tank installation structure comprises a water tank (10), an installation space (21), and a connection structure; the water tank (10) is arranged to fit into and out of the installation space (21) in a rotational manner; the connection structure is arranged to connect the water tank (10) to the installation space (21) when the water tank (10) is rotated into the installation space (21), and release the water tank (10) from the installation space (21) when the water tank (10) is disengaged from the installation space (21). The water tank installation structure simplifies the installation of the water tank (10), and facilitates the filling of the water tank (10) with water.

### FIELD

The present disclosure relates to the field of ventilation therapy, in particular to a water tank installation structure and a ventilation therapy device including the water tank installation structure.

### BACKGROUND

A ventilation therapy device, such as a ventilator, usually comprises a main machine, a humidifier, and a respiratory mask, wherein an inhalable air generated by the main machine is warmed and humidified by the humidifier, and then is supplied to the respiratory mask for the patient to inhale. By warming and humidifying the inhalable air with the humidifier, possible secondary infection of the respiratory tract and irritation on the cardiopulmonary system of the patient incurred by mechanical ventilation can be prevented or reduced, the pulmonary alveoli can be kept in a humid state, the activity of the pulmonary alveoli can be enhanced, the air exchange can be promoted, the consumption of the heat and the moisture content in the respiratory tract can be reduced, sputum scabs can be prevented in the respiratory tract, the viscosity of the secreta can be decreased, sputum excretion can be promoted, respiratory tract clogging can be prevented, and the compliance of the patient to the mechanical ventilation can be improved.

In the prior art, a humidifier is usually mounted in either of the two following ways: in the first way, the humidifier is arranged separately from the main machine, has an openable shell and a cavity in the shell, and a water tank is mounted in the cavity via an installation structure; in a second way, the humidifier is integrated with the main machine, i.e., the shell of the humidifier is integrated on the main machine, and a water tank is mounted in the main machine via an installation structure.

However, in either of the above ways, the water tank installation structure is complex, and the fitting relation is cumbersome. During use, the water tank has to be taken out for water replenishment frequently; however, the connection of the water tank to the main machine or shell is complex, and complex operations, such as cover opening and unlocking, etc. are required to take out the water tank, resulting in frequent operations of the patient and poor usability of the water tank.

### SUMMARY

In view of the above problems, the present disclosure provides a water tank installation structure and a ventilation therapy device including the water tank installation structure, to simplify the installation of a water tank and facilitate water replenishment into the water tank.

To attain the above object, in an aspect, the present disclosure provides a water tank installation structure, which comprises a water tank, an installation space, and a connection structure, wherein the water tank is configured to fit into and out of the installation space in a rotational manner, the connection structure is configured to connect the water tank to the installation space when the water tank is rotated into the installation space and release the connection of the water tank from the installation space when the water tank is detached from the installation space.

Optionally, the installation space has a top opening and a side opening, which are used for installation the water tank into the installation space.

Optionally, the water tank installation structure comprises a first bottom wall and a first side wall that are used to define the installation space, the water tank comprises a liquid storage cavity, and a second bottom wall and a second side wall that are used to define the liquid storage cavity; the second bottom wall corresponds to the first bottom wall and the second side wall corresponds to the first side wall when the water tank is mounted into the installation space.

Optionally, the connection structure comprises a first connection component and a second connection component that are fitted with each other, the first connection component is arranged on one of the first side wall and the second side wall, and the second connection component is arranged on the other of the first side wall and the second side wall.

Optionally, the first connection component is on an upper part of the first side wall or the second side wall, and the second connection component is on an upper part of the first side wall or the second side wall.

Optionally, the first connection component is arranged on the first side wall, and the second connection component is arranged on the second side wall.

Optionally, the first connection component comprises a recessed cavity outwardly recessed from an inner wall surface of the first side wall and a snap-fit groove arranged on an inner surface of the recessed cavity, the second connection component comprises a boss outwardly protruding from an outer wall surface of the second side wall and a snap-fit piece arranged on the boss, the boss matches the recessed cavity, the snap-fit piece matches the snap-fit groove, the snap-fit piece is configured to be snap-fitted in the snap-fit groove when the boss is received in the recessed cavity, and can be detached from the snap-fit groove when the boss is subjected to an outwardly pulling force.

Optionally, the second connection component comprises a compression spring, the boss has a cavity therein for accommodating the compression spring and the snap-fit piece, the boss has a hole in communication with the cavity, the snap-fit piece is configured to at least partially extend out of the hole under an abutting action of the compression spring, and can retract into the cavity by compress the compression spring under pressure.

Optionally, the size of the hole is smaller than the size of the snap-fit piece.

Optionally, the snap-fit piece is a sphere, and the snap-fit groove is a hemispherical groove.

Optionally, the first bottom wall comprises a downwardly recessed arc-shaped area, the second bottom wall forms a downwardly protruding arc-shape that matches the arc-shaped area.

Optionally, the arc-shaped area is a heating area for heating the water tank.

Optionally, the water tank installation structure comprises a first air inlet tube, a first air outlet tube, a second air inlet tube and a second air outlet tube, the first air inlet tube is coaxially inserted in the first air outlet tube and spaced from the first air outlet tube by certain radial spacing to form a first air outflow channel, and one end port of the first air inlet tube and the first air outlet tube is located on the first side wall; the second air inlet tube is coaxially inserted in the second air outlet tube and spaced from the second air outlet tube by certain radial spacing to form a second air outflow channel, one end port of the second air inlet tube and the second air outlet tube is located on the second side wall, and the other end port of the second air inlet tube and the second air outlet tube extends into the liquid storage cavity, and the one end port of the first air inlet tube and the first air outlet tube is communicated with the one end port of the second air inlet tube and the second air outlet tube in a hermetically sealed manner via a sealing element.

Optionally, the one end port of the second air inlet tube and the second air outlet tube is on the upper part of the second side wall.

Optionally, the length of the second air inlet tube is greater than the length of the second air outlet tube, the other end port of the second air inlet tube is formed as an inclined port, a wall of the water tank opposite to the inclined port is formed as an inclined wall, so that the air from the inclined port is deflected toward a lower middle area of the liquid storage cavity.

In another aspect, the present disclosure provides a ventilation therapy device, which comprises a main machine and the water tank installation structure described above, wherein the installation space is arranged on the main machine.

With the technical scheme, in the water tank installation structure in the present disclosure, the water tank is configured to fit into and out of the installation space in a rotational manner, the connection structure is configured to connect the water tank to the installation space when the water tank is rotated into the installation space and release the connection of the water tank from the installation space when the water tank is detached from the installation space; thus, the water tank can be mounted and removed more conveniently while ensuring that the water tank is mounted in the installation space reliably; to replenish water, the water tank may be removed from the installation space by rotation to prevent water leakage into the installation space, thereby ensure the high efficiency and safety of water replenishment.

Other features and advantages of the present disclosure will be further detailed in the following embodiments.

### BRIEF DESCRIPTION OF THE DRAWINGS

The accompanying drawings are provided herein to facilitate further understanding on the present disclosure and constitute a part of this document. They are used in conjunction with the following embodiments to explain the present disclosure, but are not intended to constitute any limitation to the present disclosure. In the figures:

FIG. 1 is a schematic structural diagram of an embodiment of the ventilation therapy device in the present disclosure, which only shows the main machine and the water tank;

FIG. 2 is an exploded view of the ventilation therapy device in FIG. 1;

FIG. 3 is an isometric view of the water tank in FIG. 2 viewed from another viewing angle;

FIG. 4 is a transverse sectional view of the ventilation therapy device in FIG. 1;

FIG. 5 is a longitudinal sectional view of the ventilation therapy device in FIG. 1;

FIG. 6 is a schematic structural diagram of the main machine in FIG. 5;

FIG. 7 is a schematic structural diagram of the water tank in FIG. 5;

FIG. 8 is a schematic diagram of retraction of the snap-fit piece in FIG. 7 into the cavity of the boss;

FIG. 9 is another longitudinal sectional view of the ventilation therapy device in FIG. 1, illustrating the air flow direction;

FIG. 10 is a schematic structural diagram of the main machine in FIG. 9;

FIG. 11 is a schematic structural diagram of the water tank in FIG. 9;

FIG. 12 is a schematic structural diagram of the sealing element in FIG. 2.

### REFERENCE NUMBERS

10—water tank, 11—liquid storage cavity, 12—second bottom wall, 13—second side wall, 131—boss, 132—cavity, 133—hole, 134—snap-fit piece, 135—compression spring, 14—second air inlet tube, 141—inclined port, 15—second air outlet tube, 16—inclined wall, 20—main machine, 21—installation space, 211—top opening, 212—side opening, 22—first bottom wall, 221—arc-shaped area, 23—first side wall, 231—recessed cavity, 232—snap-fit groove, 233—guiding bevel, 24—first air inlet tube, 25—first air outlet tube, 26—sealing element, 261—inner ring, 262—outer ring, 263—connection rib.

### DETAILED DESCRIPTION

Some embodiments of the present disclosure will be detailed below with reference to the accompanying drawings. It should be understood that the embodiments described herein are only provided to describe and explain the present disclosure, but are not intended to constitute any limitation to the present disclosure.

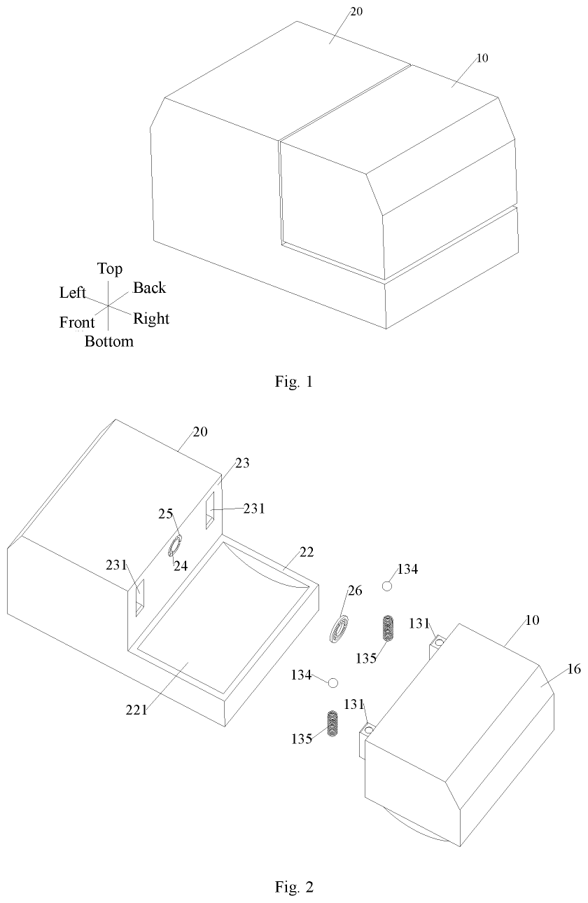

Unless otherwise specified in the present disclosure, the terms that denotes the directions or orientations, such as “top”, “bottom”, “front”, “back”, “left”, “right”, etc., refer to the directions or orientations as indicated in FIG. 1; “inside” and “outside” usually refers to inside and outside with respect to the outlines of the components.

In an aspect, the present disclosure provides a water tank installation structure, which comprises a water tank 10, an installation space 21, and a connection structure, wherein the water tank 10 is configured to fit into and out of the installation space 21 in a rotational manner, the connection structure is configured to connect the water tank 10 to the installation space 21 when the water tank 10 is rotated into the installation space 21, and release the connection of the water tank 10 from the installation space 21 when the water tank 10 is detached from the installation space 21.

With the technical scheme, in the water tank installation structure in the present disclosure, the water tank 10 is configured to fit into and out of the installation space 21 in a rotational manner, the connection structure is configured to connect the water tank 10 to the installation space 21 when the water tank 10 is rotated into the installation space 21 and release the connection of the water tank from the installation space 21 when the water tank 10 is detached from the installation space 21; thus, the water tank 10 can be mounted and removed more conveniently while ensuring that the water tank 10 is mounted in the installation space 21 reliably; to replenish water, the water tank 10 may be removed from the installation space 21 by rotation to prevent water leakage into the installation space 21, thereby ensure the high efficiency and safety of water replenishment.

The installation space 21 has a top opening 211 and a side opening 212, which are used for mounting the water tank 10 into the installation space 21. Thus, the water tank 10 can be mounted flexibly, and is in an exposed state after it is mounted, so that water replenishment into the water tank 10 can be carried out conveniently.

During the mounting, the water tank 10 may be rotated from the top opening 211 and the side opening 212 into the installation space 21, while the water tank 10 is connected by the connection structure to the installation space 21. To replenish water into the water tank 10, it is unnecessary to remove the water tank 10 and directly add water through the exposed water inlet located on the top or side of the water tank 10; alternatively, the water tank 10 may be removed from the installation space 21 by rotation and then water can be replenished into the water tank 10. Thus, the mounting of the water tank can be greatly simplified, water can be replenished into the water tank conveniently, and the connection structure is available in a wide range of choice, which is to say, an appropriate detachable connection structure may be used.

It should be noted that the water tank 10 and the installation space 21 may be in any appropriate shape, and the side opening 212 may be arranged appropriately depending on the shape of the installation space 21. For example, in the embodiment shown in FIGS. 1-11, the installation space 21 and the water tank 10 are generally square, and the side opening 212 covers the front side, back side and right side of the installation space 21. In other embodiments, the side opening 212 may at least cover the right side of the installation space 21, so as to provide an installation passage for the water tank 10 while the front side and/or the back side of the installation space 21 can limit the position of the water tank 10 to some extent.

In the present disclosure, as shown in FIGS. 1, 2 and 5, the water tank installation structure may further comprise a first bottom wall 22 and a first side wall 23 that are used to define the installation space 21, the water tank 10 comprises a liquid storage cavity 11, and a second bottom wall 12 and a second side wall 13 that are used to define the liquid storage cavity 11; when the water tank 10 is mounted into the installation space 21, the second bottom wall 12 corresponds to the first bottom wall 22, and the second side wall 13 corresponds to the first side wall 23. In the above arrangement, the water tank 10 can be supported and mounted. The first bottom wall 22 is configured to support the water tank 10, the connection structure may be arranged on the bottom walls 22, 12 or on the side walls 23, 13, preferably is arranged on the side walls 23, 13.

To facilitate mounting the water tank 10 by rotation and realize a connection to the installation space 21 while the water tank 10 is mounted, the first bottom wall 22 may comprise a downwardly recessed arc-shaped area 221, the second bottom wall 12 may be formed in a downwardly protruding arc shape that matches the arc-shaped area 221, and the connection structure may be arranged on the second side wall 13 and the first side wall 23.

The second side wall 13 and the first side wall 23 may have shapes that match each other respectively; for example, both the second side wall 13 and the first side wall 23 may be flat surfaces or curved surfaces, and may be complementary to each other in shape. In the case that the second side wall 13 and the first side wall 23 are complementary to each other in shape, for example, the second side wall 13 and the first side wall 23 have a plurality of bulges and recesses that match each other, the water tank 10 can be removably mounted in the installation space 21 by means of the complementary shapes of the second side wall 13 and the first side wall 23; in such a case, the connection structure comprises a plurality of matching bulges and recesses formed on the second side wall 13 and the first side wall 23 respectively. In the case that both the second side wall 13 and the first side wall 23 are flat surfaces or curved surfaces, matching connection components may be arranged on the second side wall 13 and the first side wall 23 to realize detachable installation.

Specifically, the connection structure may comprise a first connection component and a second connection component that are fitted with each other, the first connection component is arranged on one of the first side wall 23 and the second side wall 13, and the second connection component is arranged on the other of the first side wall 23 and the second side wall 13.

In order to improve the stability, reliability and flexibility of the installation of the water tank 10, the first connection component is on an upper part of the first side wall 23 or the second side wall 13, and the second connection component is on an upper part of the first side wall 23 or the second side wall 13. That is to say, the first connection component and the second connection component are spaced away from the second bottom wall 12 and the first bottom wall 22.

In the present disclosure, the first connection component and the second connection component may be any component or structure that can realize a detachable connection. For example, the first connection component and the second connection component may be magnetic components that attract each other.

Specifically, according to an embodiment of the present disclosure, as shown in FIGS. 2-6, the first connection component is arranged on an upper part of the first side wall 23, and the second connection component is arranged on an upper part of the second side wall 13. The first connection component comprises a recessed cavity 231 outwardly recessed (i.e., in the leftward direction in FIG. 5) from the inner wall surface of the first side wall 23 and a snap-fit groove 232 arranged on the inner surface of the recessed cavity 231, the second connection component comprises a boss 131 outwardly protruding (i.e., in the leftward direction in FIG. 5) from the second side wall 13 and a snap-fit piece 134 arranged on the boss 131, the boss 131 matches the recessed cavity 231, the snap-fit piece 134 matches the snap-fit groove 232, the snap-fit piece 134 is configured to be snap-fitted in the snap-fit groove 232 when the boss 131 is received in the recessed cavity 231, and can be detached from the snap-fit groove 232 when the boss 131 is subjected to an outwardly pulling force (i.e., a force for rotating the water tank 10 out of the installation space 21).

The snap-fit piece 134 may be snap-fitted in the snap-fit groove 232 and detached from the snap-fit groove 232 by means of its deformation; for example, the snap-fit piece 134 may be a protrusion formed on the boss 131 in a protruding manner. In order to improve the reliability of the fitting between the snap-fit piece 134 and the snap-fit groove 232 and prolong the service life of the snap-fit piece 134 and the snap-fit groove 232, preferably the snap-fit piece 134 is arranged to be snap-fitted with the snap-fit groove 232 and detached from the snap-fit groove 232 by movement.

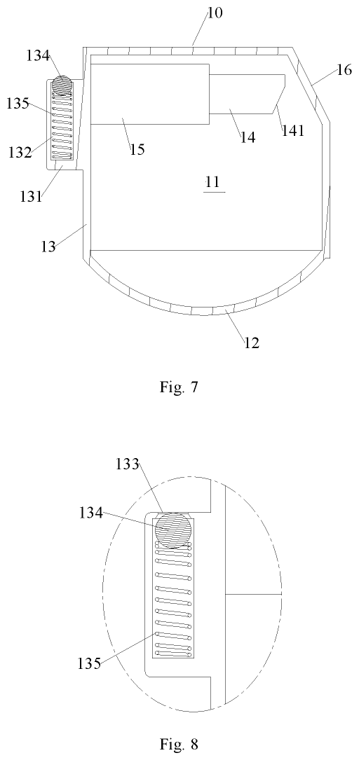

Specifically, in the embodiment shown in FIGS. 2-8, the second connection component further comprises a compression spring 135, the boss 131 has a cavity 132 for receiving the compression spring 135 and the snap-fit piece 134, and has a hole 133 in communication with the cavity 132; the snap-fit piece 134 is configured to at least partially extend out of the hole 133 (as shown in FIG. 7) under an abutting action of the compression spring 135, and can retract into the cavity 132 (as shown in FIG. 8) by compressing the compression spring 135 under pressure. That is to say, the snap-fit piece 134 has a first state in which it at least partially extends out of the hole 133 and a second state in which it retracts into the cavity 132; in the first state, the snap-fit piece 134 can be snap-fitted in the snap-fit groove 232; in the second state, the snap-fit piece 134 can make way for the boss 131 to enter or exit the recessed cavity 231.

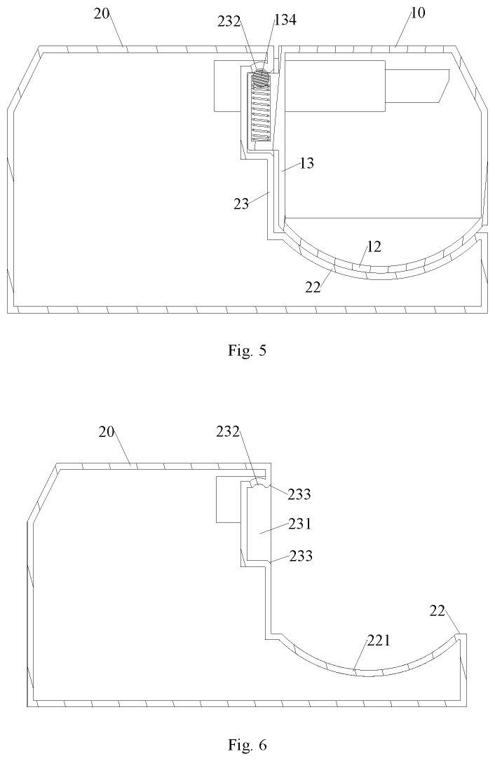

To facilitate the boss 131 to enter or exit the recessed cavity 231 quickly and successfully and further improve the convenience in the mounting and removal of the water tank 10, as shown in FIG. 6, two guiding bevels 233 may be arranged on the right upper and lower ends of the recessed cavity 231, so as to guide the boss 131 to enter or exit the recessed cavity 231.

In addition, as shown in FIG. 7, preferably the compression spring 135 is arranged in the vertical direction, the snap-fit piece 134 is located above the compression spring 135, the hole 133 is arranged at the top of the boss 131, and the snap-fit groove 232 is arranged at the top of the recessed cavity 231.

During the mounting, when the water tank 10 is loaded into the installation space 21 by rotation and the boss 131 is pushed into the recessed cavity 231, the guiding bevel 233 squeezes the snap-fit piece 134 to move downward to retract into the cavity 132 while it guides the boss 131 to enter the recessed cavity 231, till the boss 131 enters into the recessed cavity 231 entirely. At that point, the snap-fit piece 134 at least partially extends out of the hole 133 under the action of the compression spring 135 and thereby is snap-fitted in the snap-fit groove 232.

To ensure that the snap-fit piece 134 can extend and retract at the hole 133 successfully, preferably hole 133 is sized to be smaller than the snap-fit piece 134, which is to way, the snap-fit piece 134 can't extend out of the hole 133 entirely; instead, the snap-fit piece 134 partially extends out of the hole 133 under an abutting action of the compression spring 135.

As shown in FIGS. 6 and 7, the snap-fit piece 134 may be a sphere, and the snap-fit groove 232 may be a hemispherical groove. In such a case, the hole 133 is a circular hole, the diameter of the hole 133 is smaller than the diameter of the snap-fit piece 134, and the size of the snap-fit groove 232 matches the size of the part of the snap-fit piece 134 that can extend out of the hole 133.

In the present disclosure, the connection structure may comprise a plurality of groups of first connection components and second connection components that match each other, so as to improve the stability and reliability of the installation of the water tank. Preferably, as shown in FIG. 2, the connection structure comprises two groups of first connection components and second connection components that match each other and arranged at an interval in the front-back direction.

In the present disclosure, as shown in FIGS. 6 and 7, by configuring the first bottom wall 22 to comprise a downwardly recessed arc-shaped area 221 and configuring the second bottom wall 12 to form into a downwardly protruding arc-shape that matches the arc-shaped area 221, the supporting area for the water tank 10 can be increased, and the position of the water tank 10 can be limited. That is to say, the second bottom wall 12 can't be detached from the installation space 21 by moving it rightward horizontally with respect to the arc-shaped area 221. In addition, in the case that the connection structure comprises two groups of first connection components and second connection components that match each other, in association with the arc-shaped area 221 and the second bottom wall 12 that are fitted with each other, three-point fitting between the water tank 10 and the installation space 21 can be realized, thereby the stability of the water tank 10 is ensured.

The arc-shaped area 221 may be configured as a heating area; for example, the first bottom wall 22 may be formed from a heating plate, so that large-area heating of the water tank 10 can be realized.

As shown in FIG. 6, the axial direction of the arc-shaped area 221 (the front-back direction in FIG. 1) may be perpendicular to the mounting direction of the water tank 10 (the left-right direction in FIG. 1, i.e., the mounting direction of the boss 131 and the recessed cavity 231). In such a case, the water tank 10 can rotate around the axis of the arc-shaped area 221 within a certain range with respect to the arc-shaped area 221, so as to realize mounting and removal by rotation, and the angle of the water tank 10 can be adjusted conveniently to make the boss 131 and the recessed cavity 231 aligned to each other when the water tank 10 is mounted.

As shown in FIG. 5, to mount the water tank 10, the water tank 10 is placed on the arc-shaped area 221 first, with the second bottom wall 12 attached to the arc-shaped area 221, then the water tank is rotated by a certain angle and pushed leftward to get close to the first side wall 23, so that the boss 131 enters the recessed cavity 231, and the snap-fit piece 134 is snap-fitted in the snap-fit groove 232; thus, the water tank 10 is mounted. To remove the water tank 10, the water tank 10 is directly pulled rightward, so that the snap-fit piece 134 moves downward and retracts into the cavity 132 under a squeezing action at the right side of the snap-fit groove 232, and the boss 131 is detached from the recessed cavity 231; thus, the water tank 10 can be removed.

In the present disclosure, as shown in FIGS. 7 and 11, the water tank 10 may comprise a second air inlet tube 14 and a second air outlet tube 15, wherein the second air inlet tube 14 is coaxially inserted in the second air outlet tube 15 and spaced from the second air outlet tube 15 by certain radial spacing to form a second air outflow channel, one end port of the second air inlet tube 14 and the second air outlet tube 15 (i.e., the port of the left end in FIG. 11) is on the second side wall 13, and the other end port of the second air inlet tube 14 and the second air outlet tube 15 (i.e., the port of the right end in FIG. 11) extends into the liquid storage cavity 11.

By configuring the second air inlet tube 14 and the second air outlet tube 15 into a nested structural form, not only the structure of the water tank can be simplified and the space in the liquid storage cavity can be saved, but also the disturbances to the air flow can be reduced; moreover, the annular second air outflow channel can condition the air flow naturally to avoid turbulence. The second air inlet tube 14 and the second air outlet tube 15 preferably are straight tube, and the second air inlet tube 14 can also serve as a water inlet tube to fill water into the water tank 10.

Preferably, the left end port of the second air inlet tube 14 and the second air outlet tube 15 is arranged on an upper part of the second side wall 13 and at a middle position of the second side wall 13 in the front-back direction, thus, the water in the water tank 10 will not flow back into the second air inlet tube 14 and the second air outlet tube 15 when the water tank 10 is tilted as a result of handling or movement, etc.

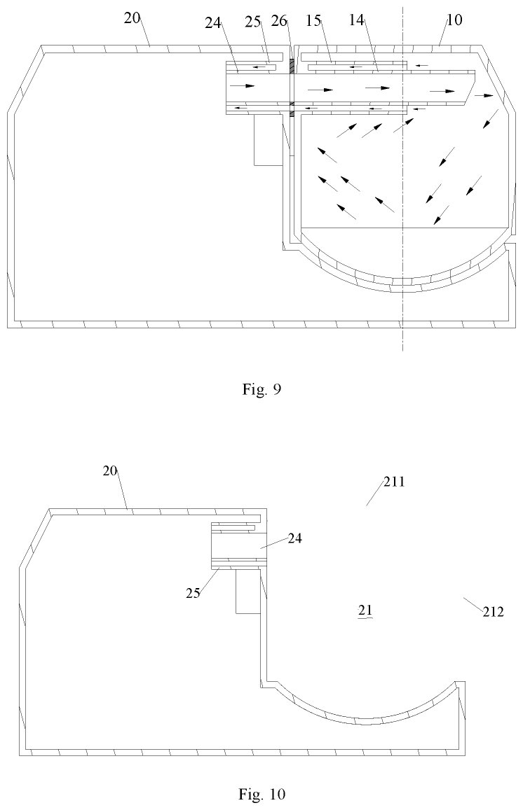

As shown in FIG. 9, preferably, the length of the second air inlet tube 14 is greater than that of the second air outlet tube 15, the right end port of the second air outlet tube 15 is at the right side of the center line of the water tank (indicated by the dotted line in FIG. 9), the right end port of the second air inlet tube 14 is formed as an inclined port 141, and the wall of the water tank 10 opposite to the inclined port 141 is formed as an inclined wall 16, so that the air from the inclined port 141 is deflected toward a lower middle area of the liquid storage cavity 11.

As indicated by the arrow in FIG. 9, after the air flows out of the second air inlet tube 14, it is deflected by the inclined wall 16 to flow toward the water surface direction, then is deflected by the water surface and other side walls of the water tank 10 for several times and enters into the second air outflow channel, and finally flows out of the second air outflow channel. The air carries warm and humid moisture content in the entire flow process, thereby a good air humidifying effect is attained. Moreover, as shown in FIG. 9, the water tank 10 has a simple internal structure without any useless part, and the air flow simply undergoes several times of deflection inside the water tank, without any turbulence.

To realize the fitting between the second air inlet tube 14 and the second air outlet tube 15, as shown in FIGS. 9-11, the water tank installation structure may further comprise a first air inlet tube 24 and a first air outlet tube 25, wherein the first air inlet tube 24 is coaxially inserted in the first air outlet tube 25 and spaced from the first air outlet tube 25 by certain radial spacing to form a first air outflow channel, one end port (i.e., the right end port in FIG. 9) of the first air inlet tube 24 and the first air outlet tube 25 is on the first side wall 23, and the right end port of the first air inlet tube 24 and the first air outlet tube 25 may be communicated with the left end port of the second air inlet tube 14 and the second air outlet tube 15 in a hermetically sealed manner via a sealing element 26. It can be understood that the first air inlet tube 24 is communicated with the second air inlet tube 14, and the first air outlet tube 25 is communicated with the second air outlet tube 15 (i.e., the first air outflow channel is communicated with the second air outflow channel).

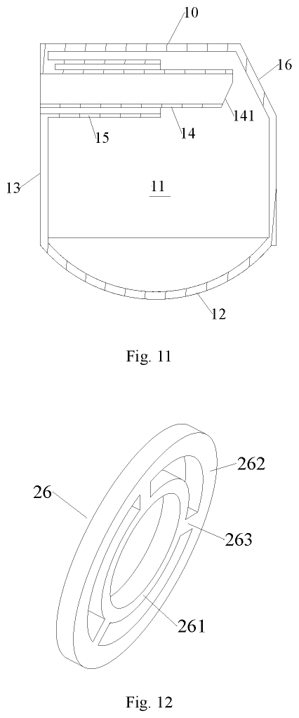

As shown in FIG. 12, the sealing element 26 is an annular member comprising an inner ring 261 and an outer ring 262 that are nested coaxially, the inner cavity of the inner ring 261 is communicated with the first air inlet tube 24 and the second air inlet tube 14, and there is certain radial spacing between the inner ring 261 and the outer ring 262 to form a communication channel for the first air outflow channel and the second air outflow channel to be communicated with each other.

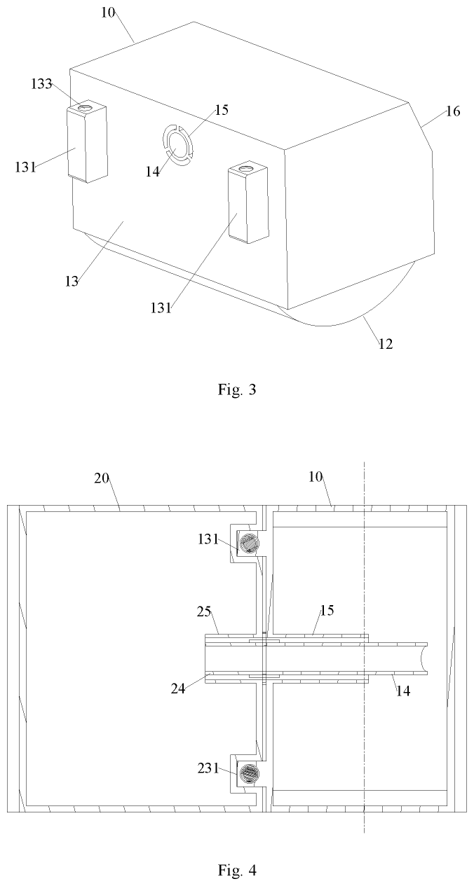

In addition, as shown in FIG. 12, the inner ring 261 and the outer ring 262 may be connected to each other via a plurality of connection ribs 263 that are spaced apart from each other. With the arrangement of the connection ribs 263, the communication channel can be separated into a plurality of arc-shaped channels, so as to attain an air flow conditioning effect. Accordingly, as shown in FIGS. 2 and 3, the second air inlet tube 14 may be connected with the second air outlet tube 15 and the first air inlet tube 24 may be connected with the first air outlet tube 25 via connecting members respectively, so that the second air outflow channel and the first air outflow channel are separated into a plurality of arc-shaped channels respectively, which correspond to the arc-shaped channels of the sealing element 26, so as to condition the out-flowing air effectively, thereby reduce pneumatic noises.

The sealing element 26 may be made of a sealing material, such as rubber, silica gel, or the like.

With the above arrangement, not only the structure can be simplified and the space can be saved, but also the disturbances to the air flow can be reduced; in addition, the annular air outflow channel can condition the air flow naturally and avoid turbulence. Moreover, since the air inlet tubes and the air outlet tubes are in a built-in form, the ports can be butt-jointed and communicated by using the sealing element 26, so that the water tank 10 can be mounted and removed independently from the air inlet tubes and the air outlet tubes. Therefore, the water tank can be mounted and removed more conveniently, and the water tank installation structure has smaller space occupation. Thus, when the water tank installation structure is applied in a ventilation therapy device, the space occupation of the ventilation therapy device can be decreased, and the esthetic appearance of the ventilation therapy device can be improved.

With the above water tank installation structure provided by the present disclosure, the user can mount and removed the water tank 10 by one-handed operation, and the water tank can be mounted, removed and filled water conveniently, thereby a good experience is achieved; the above water tank installation structure has further advantages including high safety and high reliability, simple structure, low failure rate, less parts, low cost, stable air flow, and high patient compliance, etc.

In another aspect, the present disclosure provides a ventilation therapy device, which comprises a main machine 20 and the water tank installation structure described above, wherein the installation space 21 is arranged on the main machine 20.

Specifically, as shown in FIGS. 1 and 2, the installation space 21 is arranged on the right side of the main machine 20, a first side wall 23 and a first bottom wall 22 for defining an installation space 21 are arranged on the main machine 20, the top surface, front surface and back surface of the water tank 10 are flush with corresponding sides of the main machine 20 after the water tank 10 is mounted in the installation space 21, thereby a higher esthetic appearance of the ventilation therapy device is achieved.

In addition, it should be noted that a fan is mounted in the left part of the main machine 20, the first air inlet tube 24 is communicated with an air outlet of the fan, and an air outlet communicated with the first air outlet tube 25 is arranged on the main machine 20. During use, the fan can draw ambient air into the main machine 20, then the air is fed into the first air inlet tube 24, and flows through the second air inlet tube 14 into the liquid storage cavity 11 of the water tank 10, wherein the air is humidified; then the air is discharged from the main machine through the second air outflow channel, the first air outflow channel and the air outlet sequentially, and then is delivered to the patient side for the patient to inhale.

In view that the main improvements in the present disclosure lie in the water tank and the installation of the water tank, and the arrangement of a fan inside the main machine belongs to a known technique in the art, the fan and the installation of the fan are not detailed here.

In the present disclosure, the ventilation therapy device may be a ventilator or an oxygen therapy apparatus, or the like.

While some preferred embodiments of the present disclosure are described above with reference to the accompanying drawings, the present disclosure is not limited to the details in those embodiments. Those skilled in the art can make various simple modifications and variations to the technical scheme of the present disclosure, without departing from the technical concept of the present disclosure. However, all these simple modifications and variations shall be deemed as falling in the scope of protection of the present disclosure.

In addition, it should be noted that the specific technical features described in the above embodiments may be combined in any appropriate form, provided that there is no conflict among them. To avoid unnecessary repetition, various possible combinations are not described specifically in the present disclosure.

Moreover, different embodiments of the present disclosure may also be combined freely as required, as long as the combinations don't deviate from the ideal of the present disclosure. However, such combinations shall also be deemed as being disclosed in the present disclosure.

1.-15. (canceled)

16. A water tank installation structure, comprising a water tank, an installation space, and a connection structure, wherein the water tank is configured to fit into and out of the installation space in a rotational manner, the connection structure is configured to connect the water tank to the installation space when the water tank is rotated into the installation space, and release the connection of the water tank from the installation space when the water tank is detached from the installation space.

17. The water tank installation structure of claim 16, wherein the installation space has a top opening and a side opening, which are used for mounting the water tank into the installation space.

18. The water tank installation structure of claim 16, wherein the water tank installation structure comprises a first bottom wall and a first side wall that are used to define the installation space, the water tank comprises a liquid storage cavity, and a second bottom wall and a second side wall that are used to define the liquid storage cavity; the second bottom wall corresponds to the first bottom wall and the second side wall corresponds to the first side wall when the water tank is mounted into the installation space.

19. The water tank installation structure of claim 17, wherein the water tank installation structure comprises a first bottom wall and a first side wall that are used to define the installation space, the water tank comprises a liquid storage cavity, and a second bottom wall and a second side wall that are used to define the liquid storage cavity; the second bottom wall corresponds to the first bottom wall and the second side wall corresponds to the first side wall when the water tank is mounted into the installation space.

20. The water tank installation structure of claim 18, wherein the first bottom wall comprises a downwardly recessed arc-shaped area, the second bottom wall forms a downwardly protruding arc-shape that matches the arc-shaped area.

21. The water tank installation structure of claim 19, wherein the first bottom wall comprises a downwardly recessed arc-shaped area, the second bottom wall forms a downwardly protruding arc-shape that matches the arc-shaped area.

22. The water tank installation structure of claim 18, wherein the connection structure comprises a first connection component and a second connection component that are fitted with each other, the first connection component is arranged on one of the first side wall and the second side wall, and the second connection component is arranged on the other of the first side wall and the second side wall.

23. The water tank installation structure of claim 20, wherein the connection structure comprises a first connection component and a second connection component that are fitted with each other, the first connection component is arranged on one of the first side wall and the second side wall, and the second connection component is arranged on the other of the first side wall and the second side wall.

24. The water tank installation structure of claim 22, wherein the first connection component is on an upper part of the first side wall or the second side wall, and the second connection component is on an upper part of the first side wall or the second side wall.

25. The water tank installation structure of claim 22, wherein the first connection component is arranged on the first side wall, the second connection component is arranged on the second side wall, the first connection component comprises a recessed cavity outwardly recessed from an inner wall surface of the first side wall and a snap-fit groove arranged on an inner surface of the recessed cavity, the second connection component comprises a boss outwardly protruding from an outer wall surface of the second side wall and a snap-fit piece arranged on the boss, the boss matches the recessed cavity, the snap-fit piece matches the snap-fit groove, the snap-fit piece is arranged to be snap-fitted in the snap-fit groove when the boss is received in the recessed cavity, and is able to be detached from the snap-fit groove when the boss is subjected to an outwardly pulling force.

26. The water tank installation structure of claim 24, wherein the first connection component is arranged on the first side wall, the second connection component is arranged on the second side wall, the first connection component comprises a recessed cavity outwardly recessed from an inner wall surface of the first side wall and a snap-fit groove arranged on an inner surface of the recessed cavity, the second connection component comprises a boss outwardly protruding from an outer wall surface of the second side wall and a snap-fit piece arranged on the boss, the boss matches the recessed cavity, the snap-fit piece matches the snap-fit groove, the snap-fit piece is arranged to be snap-fitted in the snap-fit groove when the boss is received in the recessed cavity, and is able to be detached from the snap-fit groove when the boss is subjected to an outwardly pulling force.

27. The water tank installation structure of claim 25, wherein the second connection component comprises a compression spring, the boss has a cavity therein for accommodating the compression spring and the snap-fit piece, the boss has a hole in communication with the cavity, the snap-fit piece is configured to at least partially extend out of the hole under an abutting action of the compression spring, and is able to retract into the cavity by compressing the compression spring under pressure.

28. The water tank installation structure of claim 27, wherein the size of the hole is smaller than the size of the snap-fit piece.

29. The water tank installation structure of claim 27, wherein the snap-fit piece is a sphere, and the snap-fit groove is a hemispherical groove.

30. The water tank installation structure of claim 20, wherein the arc-shaped area is a heating area for heating the water tank.

31. The water tank installation structure of claim 18, wherein the water tank installation structure comprises a first air inlet tube, a first air outlet tube, a second air inlet tube and a second air outlet tube, the first air inlet tube is coaxially inserted in the first air outlet tube and spaced from the first air outlet tube by certain radial spacing to form a first air outflow channel, and one end port of the first air inlet tube and the first air outlet tube is located on the first side wall; the second air inlet tube is coaxially inserted in the second air outlet tube and spaced from the second air outlet tube by certain radial spacing to form a second air outflow channel, one end port of the second air inlet tube and the second air outlet tube is located on the second side wall, and the other end port of the second air inlet tube and the second air outlet tube extends into the liquid storage cavity, and the one end port of the first air inlet tube and the first air outlet tube is communicated with the one end port of the second air inlet tube and the second air outlet tube in a hermetically sealed manner via a sealing element.

32. The water tank installation structure of claim 19, wherein the water tank installation structure comprises a first air inlet tube, a first air outlet tube, a second air inlet tube and a second air outlet tube, the first air inlet tube is coaxially inserted in the first air outlet tube and spaced from the first air outlet tube by certain radial spacing to form a first air outflow channel, and one end port of the first air inlet tube and the first air outlet tube is located on the first side wall; the second air inlet tube is coaxially inserted in the second air outlet tube and spaced from the second air outlet tube by certain radial spacing to form a second air outflow channel, one end port of the second air inlet tube and the second air outlet tube is located on the second side wall, and the other end port of the second air inlet tube and the second air outlet tube extends into the liquid storage cavity, and the one end port of the first air inlet tube and the first air outlet tube is communicated with the one end port of the second air inlet tube and the second air outlet tube in a hermetically sealed manner via a sealing element.

33. The water tank installation structure of claim 31, wherein the one end port of the second air inlet tube and the second air outlet tube is located on the upper part of the second side wall.

34. The water tank installation structure of claim 31, wherein the length of the second air inlet tube is greater than the length of the second air outlet tube, the other end port of the second air inlet tube is formed as an inclined port, a wall of the water tank opposite to the inclined port is formed as an inclined wall, so that the air from the inclined port is deflected toward a lower middle area of the liquid storage cavity.

35. A ventilation therapy device, comprising a main machine and the water tank installation structure of claim 31, wherein the installation space is arranged on the main machine.
